(19) United States
(12) Patent Application Publication
(10) Pub. No.: US 2006/0089588 A1
(43) Pub. Date: Apr. 27, 2006

(54) Aortic balloon occlusion cannula

(76) Inventor: Albertus Scheule, Tubingen (DE)

Correspondence Address: CHRISTIE, PARKER & HALE, LLP, PO BOX 7068, PASADENA, CA, 91109-7068, US

(21) Appl. No.: 11/179,708

(22) Filed: Jul. 11, 2005

Related U.S. Application Data
(63) Continuation-in-part of application No. 10/099,496, filed on Mar. 13, 2002, now Pat. No. 6,926,689.

(30) Foreign Application Priority Data
Sep. 13, 1999 (EP) 99117905.2

(51) Int. Cl. A61M 37/00

(52) U.S. Cl. 604/6.16

(57) ABSTRACT

An aortic balloon occlusion cannula for the occlusion of the ascending aorta during cardiac surgery includes a cannula containing several lumina that are separated from one another. The cannula carries two dilatable occlusion balloons positioned at a distance from each other, one of the balloons being neighbored to the proximal end of the cannula which faces the heart and each of the balloons being connected to its own lumen which enables its dilation in independence from the other balloon. The cannula additionally contains at least one further lumen that is connected on the proximal side of the proximal occlusion balloon and on the distal side of the distal occlusion balloon to the lumen of the aorta and is adapted to be connected to an extracorporal blood supply device. A valve is assigned to the part of this additional lumen which ends on the proximal side of the proximal occlusion balloon.

### CROSS-REFERENCE TO RELATED APPLICATION

This patent application is a continuation-in-part application of allowed U.S. patent application Ser. No. 10/099,496, filed Mar. 13, 2002, the disclosure of which is incorporated fully herein by reference.

### BACKGROUND

The invention concerns an aortic balloon occlusion cannula for the occlusion of the ascending aorta during cardiac surgery.

An arteriosclerotically altered ascending aorta represents a problem in the field of cardiac surgery. It occurs in almost all patients who suffer from calcification of the coronary vessels or, to different degrees, in patients with valvular diseases. It is necessary to insert an aortic cannula into the ascending aorta in order to connect for instance a patient, who undergoes a bypass operation, to the extra corporal circulation (heart-lung machine). The blood circulation is separated from the heart by clamping the ascending aorta towards the heart that is proximal, with a metal clamp, which is attached at right angles. There is, however, the danger of a detachment of particles or plaques from the wall of the ascending aorta which are transported by the blood flow especially into the blood vessels of the head and therefore into the brain. This leads to embolies, which appear clinically often in form of neurological failures (cerebral infarction).

An aortic balloon occlusion cannula is known from the DE 19 15 933 A1. It is used to avoid the risks going along with aortic clamping at right angles during the extracorporeal circulation that is applied during cardiac surgeries. It includes an occlusion cannula that can be inserted into a catheter. Its lumen is connected on both sides to a dilatable balloon, which allows closing the ascending aorta from the inside by means of a balloon occlusion during the ischemic time without an aortic clamping at right angles. A similar aortic occlusion cannula has also been described in U.S. Pat. No. 5,334,142, especially in connection with cardiopulmonary resuscitation. Further embodiments of occlusion cannulae having two dilatable balloons are disclosed in U.S. Pat. No. 5,458,574 and in EP 1 086 717 A1.

These balloon cannulae, however, do not solve the problems of other dangers, which can also lead to a calcified embolie as a result of an arteriosclerotic ascending aorta.

In order to be able to suture the vein bypass to the aorta during a bypass surgery, the aorta has to be clamped with a metal clamp over a certain length in the area of the suture. There is a considerable risk of embolie connected with this therapy. In order to provoke a cardiac arrest, it is also necessary to infuse a cardioplegic substance into the ascending aorta. It is also possible that plaques are separated if a cardioplegic conduction is inserted especially for this purpose into the ascending aorta.

### SUMMARY

An object of this invention is to offer a solution to the above mentioned problem and to create an aortic balloon occlusion cannula for the occlusion of the ascending aorta during surgeries. This aortic balloon occlusion cannula is adapted to reduce the danger of the separation of calcified plaques from the calcified ascending aorta and to guarantee a careful treatment of the aorta during the surgery.

The new aortic cannula can be placed into the aorta via the lefthand ventricle. Thus the calcified aorta needs not to be opened and subsequently reclosed. It is just with an arteriosclerotic aorta ascendens that suturing of the aorta can be a problem which occasionally necessitates a partial clamping of this area resulting in an increased risk of embolies caused by calcified particles.

Furthermore, the new aortic cannula permits the clamping of the ascending aorta from the inside by means of a dilatable occlusion balloon. In addition, the cannula carries a second occlusion balloon that is positioned in a certain distance from the first balloon. It is able to separate an area from the perfusion. This area is determined by the distance between the two balloons. It serves then for the suture of the vein bypasses. With this, the dangerous tangential clamping of an aortic area is avoided. In the meantime, the aortic root perfusion can retrogradely be performed by means of a coronary sinus in order to reduce the ischemic time.

If the aortic root perfusion is retrogradely applied via the coronary sinus the new cannula can furthermore assume the function of sucking off the aortic root perfusion. It is one of the advantages of the new aortic cannula that, in contrast to the above discussed state of the prior art, the occlusion cannula need not be inserted through the calcified aorta and that, furthermore, the aorta need not be clamped from its outside, neither tangentially nor transversely. In addition, neither the tubing for the aortic root perfusion nor a suction tube need to be additionally inserted into the aorta through a separate further access in the aorta. Finally, the field of the surgical operation remains much clearer as it is the case with the usual technique, for the reason that there are less additional tubings and clamps that tend to encumber the operation field.

### BRIEF DESCRIPTION OF THE DRAWINGS

The illustration shows an example of the subject of the invention. The following figures show:

FIG. 1 is a balloon cannula according to the invention in situ in a schematic illustration;

FIG. 2 is the cannula according FIG. 1 cut lengthways along the line II-II of FIG. 1 in a schematic illustration; and

FIG. 3 is the balloon occlusion cannula according to FIG. 1 in an axial cut, in a side view and in a very simplified illustration.

FIG. 4 is a second embodiment of a balloon occlusion cannula according to the invention, illustrated in an axial cut, in a side view and in a very simplified illustration similar to FIG. 3.

FIG. 5 is the cannula according to FIG. 4 in a cross-sectional view similar to FIG. 2.

FIG. 6 is a further embodiment of a balloon occlusion cannula according to the invention, in a side view and in a very simplified illustration.

FIG. 7 is a cross-sectional perspective view of the cannula of FIG. 6 and

FIG. 8 is an illustration of an application of the cannula of FIG. 4 to place an endovascular prothesis in an aorta.

### DETAILED DESCRIPTION

The aortic balloon occlusion cannula, which is generally marked by a 1, is to occlude the ascending aorta indicated at 2, during cardiac surgeries. It contains a cannula tube 3 that is made out of an elastic material. This material enables the introduction of the cannula tube 3, while adapting to the required curvatures, into the ascending aorta 2 through a corresponding incision at 4 in the lefthand cardiac ventricle. When inserting the cannula care must be taken to pass the valvola aortae 20 as carefully as possible. Therefore, in the beginning a guide wire is placed into the heart and subsequently the cannula is inserted by means of a guide rod that is located in the cannula tube 3 and provided with a tapered end. This guide rod defines a lumen for said guide wire and is removed from the cannula tube 3 when the cannula is correctly inserted.

Cannula tube 3 can also be pre-shaped according to the curvature of the ascending aorta. Two dilatable occlusion balloons 5 and 6 that are positioned in a distance from each other, are mounted on the cannula tube 3. The first balloon 5 is positioned at the proximal end of the cannula tube 3, facing the heart, while the other balloon 6 ought to be in a distance of approximately 20 to 30 mm from the balloon 5, closer to the distal end of the cannula tube 3, facing the body.

The areas of the outer surface of the cannula tube 3 close to the two occlusion balloons 5, 6 are tapered or frustro-conically shaped at 50 and 60, respectively, or the cannula tube 3 is provided with a frustro-conical headpiece, which means enhance a smooth passage of the valvola aortae 20 when the cannula tube 3 is inserted or removed. For a similar reason the end portions 30 of the cannula tube 3 can be tapered to a point.

Both occlusion balloons 5, 6 consist of an elastic dilatable plastic, e.g. polyethylene which provides sufficient stiffness and consistency of shape in order to guarantee a secure closing of the ascending aorta 2. The diameter of the two balloons 5, 6 is adapted to the inner diameter of the ascending aorta 2 and its size is about 35-45 mm. The axial width of each of the two occlusion balloons is about 1.5 to 2 cm or more. Both occlusion balloons 5, 6 can be positioned on the cannula tube 3 either fixed or in such a way that they are movable towards each other in order to enable an adaption to the anatomic situation in each individual case.

Furthermore the two occlusion balloons can have different axial widthwise dimensions, the occlusion balloon 6 located close to the end being wider than the proximal occlusion balloon 5.

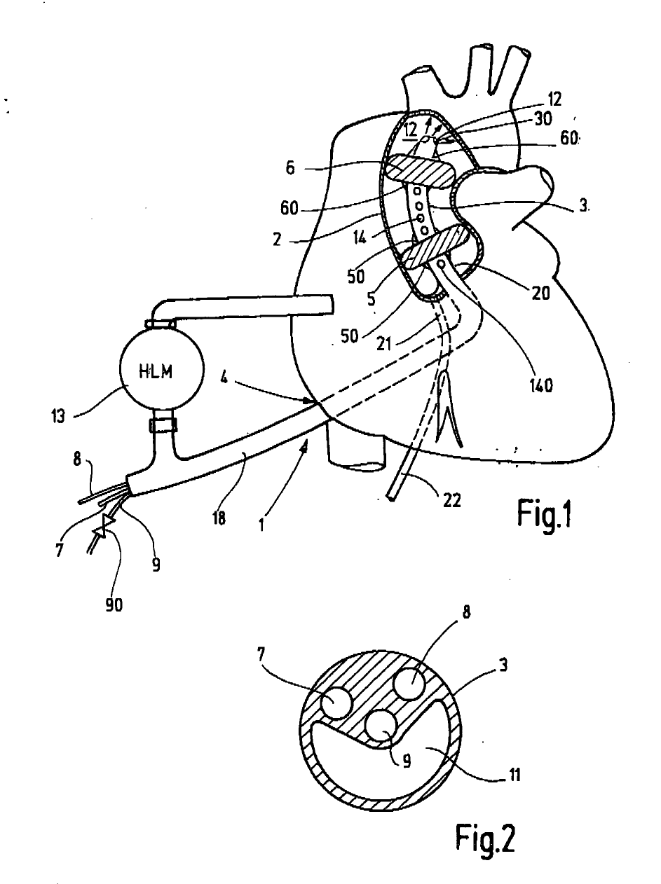

The cannula 1 contains several separate lumina. They form independent conduits and can be separated from one another as for instance indicated schematically in FIG. 2.

A first lumen 7, indicated in FIG. 3 by a chain dotted line, leads to the first occlusion balloon 5 and permits its dilatation by means of a suitable dilatation liquid (physiologic salt solution). A second lumen 8, shown in FIG. 3 by a double chain dotted line, leads to the second occlusion balloon 6 and enables its dilatation by means of the corresponding dilatation liquid.

Outside of cannula tube 3, suitable fittings for the supply of dilatation liquid, shut-off valves and controls are assigned to the conduits formed by the lumina 7, 8 in order to dilate the occlusion balloons 5, 6 during the occlusion of the ascending aorta 2, and to return them into the non-dilated state. These means and devices are not illustrated in detail. They are well-known.

A third lumen 9 defines a conduit that opens via openings 140 on the proximal side, facing the heart, of the occlusion balloon 5. Said conduit is provided with additional openings 14 in the section between the two occlusion balloons 5 and 6. With the cannula 1 inserted and with an operative valvola aortae 20, liquid can be sucked-off the portion of the aorta ascendens 2 which is proximal to the occlusion balloon 5 through said openings 14. Furthermore, said conduit permits to suck liquid off the area between the two occlusion balloons and 6 if the occlusion balloon 5 is not dilated or in case of a potential leakage of the occlusion balloon 5. The conduit defined by lumen 9 contains a shut-off organ 90 that permits the control of the supply of the heart protecting solution as required. The internal diameter of the lumen 9 is approximately 3 mm (to give an approximate size). The liquid rate for a retrograde myocardial perfusion is in the order of 500 ml per minute, at a maximum. Additional openings may be added to the third lumen which open to the left ventricle for venting. Alternatively, a separate lumen (not shown) may be included in the cannula that opens to the left ventricle for venting, if necessary.

A wider and larger lumen 11 is enclosed by the cannula tube 3. It is connected to the lumen of the distal part, i.e. the side facing the body, of the aorta ascendens when the cannula is inserted in the aorta 2 over one or more opening(s) 12 in the wall of the cannula 1. Lumen 11 forms a blood conduit that is, as indicated schematically in FIG. 1, connected to a heart-lung machine 13 which maintains the circulation extracorporally. The diameter of each of the openings 12 is 10 mm or more.

When the new cannula is used—this cannula can also be named as an aortic endoclamping cannula with double balloon technique and integrated cardioplegic cannula—at first the distal occlusion balloon 6 is dilated after the insertion of the cannula 1 in the aorta ascendens 2 through the incision 4 in the lefthand ventricle.

With the cannula 1 inserted and with the valvola aortae 20 being in an operative state a cardioplegic solution can be supplied to the heart through a coronary sinus 21 and a conduit 22 that is in the form of a coronary sinus catheter. Said cardioplegic solution is suctioned off through the conduit defining the lumen 9 while the body circulation is supplied with blood from the heart-lung machine 13 over the lumen 11 and the opening(s) 12. At this time the anastomoses located close to the heart are sutured.

Subsequently the second occlusion balloon 5 is dilated, so that the aorta ascendens 2 is additionally occluded at this place. The section 17 of the aorta ascendens 2 delimited between the two occlusion balloons 5 and 6 is opened and provided with “punched out” anastomotic holes, where upon the anastomoses are sutured. One of them is shown in FIG. 3 at 19. During these proceedings blood is supplied to the heart through the coronary sinus catheter 22. The blood is sucked-off through the conduit defining the lumen 9.

Upon the termination of these measures, the two occlusion balloons 5 and 6 are deflated, and the organism is trained to function without the heart-lung machine. The cannula 1 is taken out of the aorta ascendens 2 through the lefthand ventricle. The cannula tube 3 shows a generally straight or, according to the aorta curved section in the section 17 between the two occlusion balloons 5 and 6. As shown, the adjacent area 18 can lead approximately at right angles from the area 17.

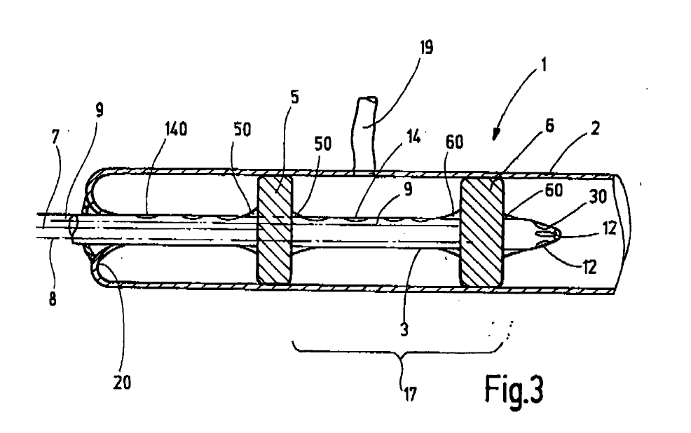

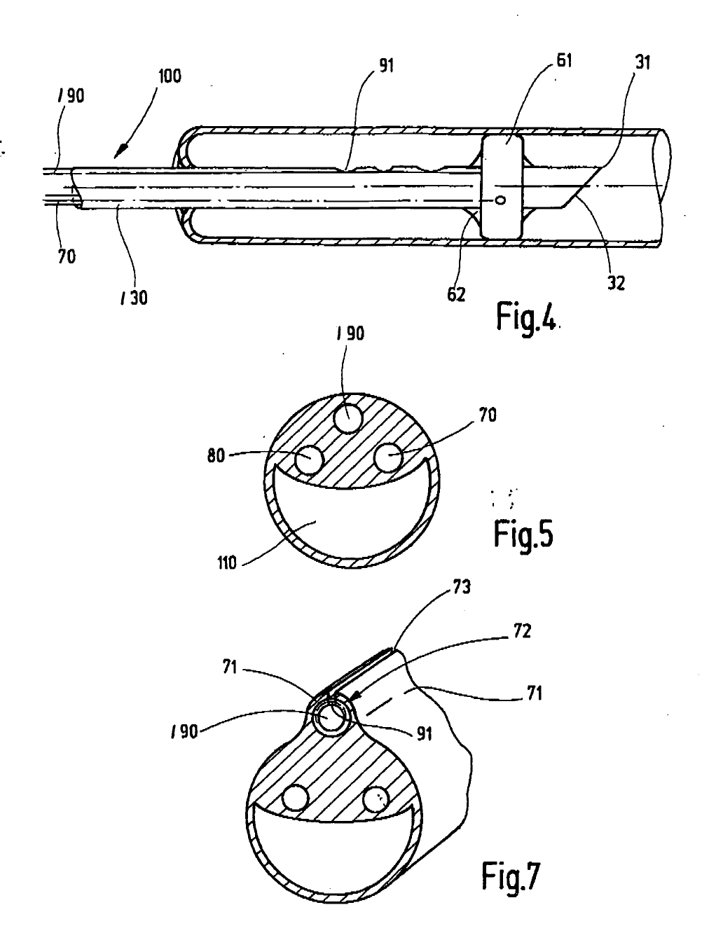

A somewhat simplified embodiment of the aortic balloon occlusion cannula according to the invention is illustrated in FIGS. 4 to 8 of the drawing and marked by a 100. It comprises a cannula tube 130 that is somewhat similar to the cannula tube 3 of the embodiment shown in FIGS. 1, 2. It is also made out of an elastic material and adapted to be introduced into the ascending aorta 2 through a corresponding incision in the lefthand cardiac ventricle. On the cannula tube 130 a dilatable occlusion balloon 61 is positioned close to the open distal end 31 of the cannula tube 130. The areas of the outer surface of the cannula tube 130 close to the occlusion balloon 61 are tapered or frustro-conically shaped as at 62, respectively. The cannula tube 130 is provided, at its open end, with a frustro-conical head piece or it is tapered as at 32 to enhance a smooth passage of the valvola aortae 20 when the cannula tube 130 is inserted or removed.

The cannula 100 comprises several separate lumina. They form independent conduits and are adapted to be connected to different apparatus at their ends remote from the balloon 61. Of these lumina a first lumen 70 leads to the occlusion balloon 61 and permits its dilatation by means of a suitable dilatation liquid. A second lumen 80 is optionally provided, for example for the supply of a heart protecting solution as required.

A third lumen 190 defines a conduit that opens via openings 91 on a side of the balloon 61 that is remote from its open end 31. When inserted into the aorta ascendens this side of the balloon is the proximal side, facing the heart. Finally, there is a wider and larger lumen 110 enclosed by the cannula tube 130. It corresponds to the wide and large lumen 11 of the FIGS. 1, 2 embodiment and serves to form a blood conduit when the cannula is inserted into the aorta.

The cannula illustrated in FIGS. 4, 5 can be used in a similar way as it is has been described in connection with the FIGS. 1, 2 embodiment of the cannula 1. For bypass surgery the cannula tube 130 is inserted through the incision 4 in the lefthand cardiac ventricle and the occlusion balloon is placed at an appropriate location within the aorta ascendens. The vent openings 91 are in such a distance from the balloon 61 that when the balloon 61 is properly located in the aorta ascendens the vent openings 91 are located within the lefthand cardiac ventricle thereby allowing the lefthand ventricle to be relieved.

Under another aspect the balloon occlusion cannula 100 can be used for occluding the aorta ascendens when an aortic dissection is to be performed. In this case a balloon 61 is used that has preferably an outer diameter in the range of about 50 to 60 mm.

Under a further aspect the aortic balloon occlusion cannula may be used for combined surgeries, e.g. cardiac surgeries to replace the arcus aortae in combination with placing of an endovascular prothesis in the aorta of a patient. This application is schematically illustrated in FIG. 8. A generally tube-shaped endovascular prothesis 65 is placed in the aorta 66. To do this the prothesis 65 is connected to the cannula tube 130 by inserting the cannula tube 130 into the prothesis 65 and by dilating the balloon 61, thereby attaching the prothesis to the cannula. By moving the cannula tube 130 the prothesis 65 can be brought to a desired location.

During all of these procedures the large lumen 110 of the cannula tube 130 allows perfusion of the lower portions of the patient's body while surgery is performed on the aorta ascendens or on the arcus aortae. For most of these applications the vent conduit 190 should reach close to the balloon 61 with vent openings 91 being located close to the balloon 61.

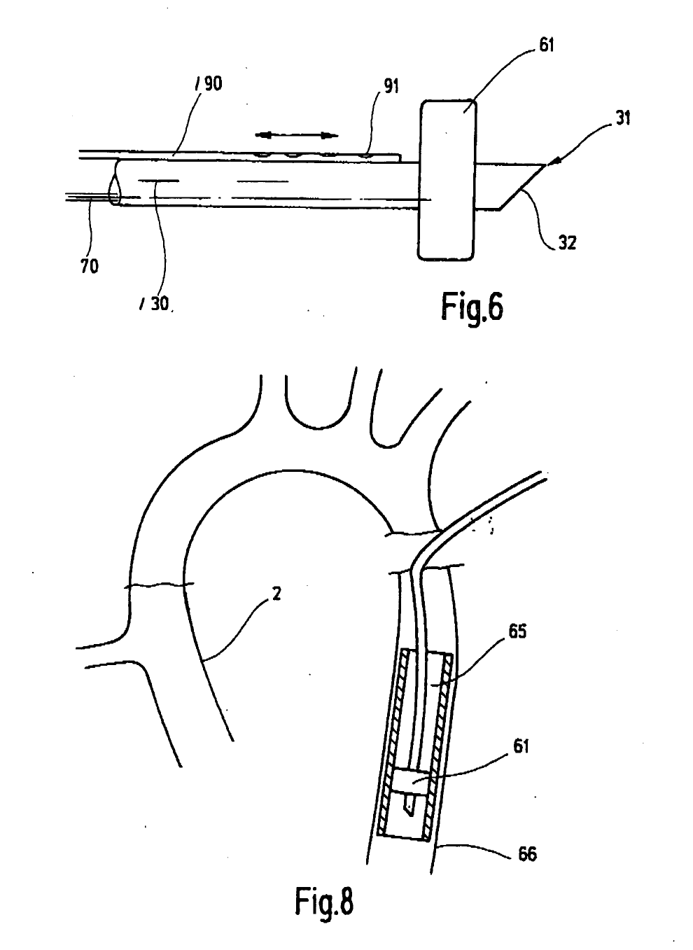

For some applications the vent openings 91 should be located in a greater distance from the balloon 61. For example, when the vent conduit 190 is used for sucking off a cardioplegic solution, vent openings 91 should be close to the balloon 61 while for other applications the vent openings 91 should be close to the tip of the heart. To avoid the necessity to provide a large number of aortic balloon occlusion cannulae having vent openings 91 that are in different distances from the balloon 61, the vent conduit can be adjustably supported on the cannula tube 130 as this is schematically illustrated in FIG. 6. The vent conduit 190 is in the form of a cylindrical tube made of an elastic material, and on the outer surface of the cannula tube 130 two parallel lips 71 are formed that enclose an inner cylindrical bore 72 in which the vent conduit 190 is received. The two lips 71 are separated from one another by a gap 73 on their radially outer side thereby frictionally clamping the vent conduit 190 between each other. The gap 73 extends over at least a portion of the lengths of the cannula tube 130 and allows the vent conduit 190 to be shifted along the length dimension of the cannula tube 130. By appropriate adjusting the vent conduit 190 with respect to the annular tube 130, vent holes 91 can be provided in each desired location along the length dimensions of the cannula tube 130.

The vent openings 91 are registered with the gap 73 thereby permitting a free fluid flow into and out of the vent openings 91.

1. An aortic balloon occlusion cannula for the occlusion of the aorta ascendens during cardiac surgeries comprising:
a cannula containing several lumina that are separated from one another, said cannula carrying a proximal dilatable occlusion balloon and a distal dilatable occlusion balloon positioned at a distance from each other, one of said balloons being neighbored to a distal end of the cannula which faces away from the heart and each of said balloons being connected to its own lumen which enables its dilatation in independence from the other balloon,
said cannula being insertable through the lefthand ventricle of the heart and through the valvola aortae and comprising at least one further lumen, said further lumen being connected on a distal side, facing the body, of the distal occlusion balloon to the lumen of the aorta and being adapted to be connected to an extracorporal blood supply device, and
an additional separate lumen provided in said cannula and forming a return conduit for liquid and opening on a proximal side of the proximal occlusion balloon.

2. The balloon occlusion cannula according to claim 1, wherein the cannula is provided in its section between the proximal and distal occlusion balloons with at least one opening that opens in this section and that is connected to the additional lumen in the cannula.

3. The balloon occlusion cannula according to claim 1, wherein the cannula comprises a cannula tube comprising a first section in the area of the proximal and distal occlusion balloons and a second section leading at right angles therefrom when said cannula is inserted in the aorta ascendens.

4. The balloon occlusion cannula according to claim 3, wherein the first section of the cannula tube is essentially straight.

5. The balloon occlusion cannula according to claim 3, wherein the first section of the cannula tube is curved according to the shape of the aorta ascendens.

6. The balloon occlusion cannula according to claim 1, wherein the proximal and distal occlusion balloons are mounted in such a way that they are movable with respect to each other in the direction of the axis of the cannula.

7. The balloon occlusion cannula according to claim 1, wherein the proximal and distal occlusion balloons have different axial width dimensions.

8. The balloon occlusion cannula according to claim 7 wherein the distal occlusion balloon has an axial width that is wider than the axial width of the proximal occlusion balloon.

9. The balloon occlusion cannula according to claim 1, wherein the cannula comprises a cannula tube having an outer wall, said outer wall being flared toward its neighboring occlusion balloon in the vicinity of at least one of the proximal and distal occlusion balloons.

10. The balloon occlusion cannula according to claim 9, wherein the frustro-conical flaring is provided on head piece means that are mounted on the essentially cylindrical cannula tube.

11. The balloon occlusion cannula according to claim 1, wherein the cannula comprises a cannula tube that is tapered on its distal end facing the body.

12-19. (canceled)

20. An aortic balloon occlusion cannula for the occlusion of the aorta during cardiac surgeries comprising:
a cannula including several lumina that are separated from one another said cannula having an open end and a dilatable occlusion balloon positioned at a distance from said open end, said balloon being connected to its own lumen to enable its dilatation,
said cannula comprising at least one further lumen forming a conduit that extends close to a side of said balloon that is remote from the open end of said cannula, said conduit having at least one vent opening leading to the exterior of the cannula and
said balloon being sized to occlude the aorta when inserted therein and dilated.

21. An aortic balloon occlusion cannula for the occlusion of the aorta ascendens during cardiac surgeries comprising:
a cannula including several lumina that are separated from one another, said cannula having an open end and a dilatable occlusion balloon positioned at a distance from said open end, said balloon being connected to its own lumen to enable its dilatation,
said cannula being insertable through the left-hand ventricle of the heart and through the valvola aorta and comprising at least one further lumen forming a conduit that extends close to a side of said balloon that is remote from the open end of said cannula, said conduit having at least one vent opening leading to the exterior of said cannula,
said balloon being sized to occlude the aorta ascendens when inserted therein and dilated.

22. The balloon occlusion cannula according to claim 21 wherein the at least one vent opening of said conduit is located in such a distance from said balloon that with the balloon being inserted into the aorta ascendens the at least one vent opening opens into the left hand ventricle of the heart.

23. The balloon occlusion cannula according to claim 20, wherein said conduit is adjustably supported on said cannula such that the distance of said at least one vent opening from said balloon can be adjusted along the length dimension of said cannula.

24. The balloon occlusion cannula according to claim 20 wherein said balloon is sized to be inserted into a tube-like endovascular prothesis and to be attached thereto by dilatation, and that said cannula is adapted to be used for placing said prothesis in a desired location in the aorta.

25. The balloon occlusion cannula according to claim 20 wherein said balloon has an outer diameter in the range of 50 to 60 cm.

26. The balloon occlusion cannula according to claim 21, wherein said conduit is adjustably supported on said cannula such that the distance of said at least one vent opening from said balloon can be adjusted along the length dimension of said cannula.

27. The balloon occlusion cannula according to claim 21 wherein said balloon is sized to be inserted into a tube-like endovascular prothesis and to be attached thereto by dilatation, and that said cannula is adapted to be used for placing said prothesis in a desired location in the aorta.
